(19) United States
(12) Patent Application Publication
(10) Pub. No.: US 2002/0022009 A1
(43) Pub. Date: Feb. 21, 2002

(54) COSMETIC OR DERMATOLOGICAL COMPOSITION COMPRISING A FILM-FORMING POLYMER AND AN AQUEOUS SILICONE EMULSION

(76) Inventors: VALERIE DE LA POTERIE, LE CHATELET EN BRIE (FR); MYRIAM MELLUL, L'HAY LES ROSES (FR); ISABELLE BARA, PARIS (FR)

Correspondence Address: Oblon Spiviak McClelland Maier & Nuestadt, 1755 Jefferson Davis Highway, Fourth Floor, Arlington, VA, 22202, US

(21) Appl. No.: 09/206,323

(22) Filed: Dec. 7, 1998

(30) Foreign Application Priority Data
Dec. 5, 1997 (FR) 97 15414

(51) Int. Cl. A61K 7/021

(52) U.S. Cl. 424/63

(57) ABSTRACT

The present application relates to a cosmetic or dermatological composition suitable for application on the skin and/or the lips, comprising at least one film-forming polymer in a dispersion and at least one aqueous silicone emulsion capable of forming a non-transferring cohesive film on the lips and/or the skin.

### BACKGROUND OF THE INVENTION

1. Field of the Invention:

The present application relates to a cosmetic or dermatological composition suitable for application on the skin and/or the lips. This composition comprises a film-forming polymer, preferably in the form of a dispersion of particles, and it can be used as a make-up product.

2. Discussion of the Background

Compositions to be applied to the skin and/or the lips such as lipstick and color bases generally are in the form of sticks, supple paste or poured paste, and comprise fatty substances such as oils, pasty compounds and/or waxes, and a particulate phase generally composed of fillers and pigments. When these compositions are applied on the skin or the lips, they have the disadvantage, however, of transferring. This means that the composition is capable of being deposited at least in part on certain substrates with which it comes into contact, such as, for example, a glass, a cup, a garment or the skin. By being deposited, the composition leaves a trace on the substrate. This leads to poor persistence of the composition on the skin and semi-mucous or mucous tissues, with the need to renew application thereof regularly. In addition, the appearance of unacceptable traces on certain garments and notably on blouse collars can dissuade certain women from using this type of make-up.

Another disadvantage of these compositions lies in the problem of migration. It has been observed, in fact, that certain compositions have tended to propagate inside creases and/or wrinkles in the skin, in the case of color bases; in the creases surrounding the lips, in the case of lipsticks; in the folds of the eyelid, in the case of eye shadows. In the case of eye shadows in particular, the appearance of streaks in the make-up, produced by eyelid movements, has also been observed. Furthermore, it has been observed that eye liners can also flow. All these phenomena produce a nonesthetic effect, which quite obviously the user wishes to avoid.

For several years, numerous cosmeticians have been interested in cosmetic compositions, especially “non-transferring” lipsticks or color base. For example, in JP A 61/65809, there were envisioned liquid compositions for “non-transferring” lipstick containing from 1 to 70% by weight of silicone resin with repeating silicate units, from 10 to 98% by weight of a volatile silicone oil and powdered fillers. Nevertheless, the film obtained on the lips after evaporation of the silicone oil has the disadvantage of becoming uncomfortable in the course of time (feeling of drying out and tugging).

From EP A 602905 there are also known “non-transferring” lipsticks containing a volatile silicone and a liquid silicone resin provided with an esterified side chain having at least 12 carbon atoms. The deposited lipstick film suffers especially from the disadvantage of lack of comfort in application, and in particular of being too dry.

In general, therefore, the combination of volatile oils with certain silicone compounds makes it possible to improve the “non-transferring” properties. Nevertheless, the films obtained after application of these compositions and evaporation of the volatiles still suffer from the disadvantage of being relatively matte, and thus lead to make-up with little gloss. In addition, the non-transferring properties are not total.

On the other hand, there have been described cosmetic make-up compositions comprising film-forming polymers in aqueous solution; but these compositions are sensitive to water and therefore cannot be applied to, in particular, the lips.

In addition, when the polymers are solubilized in alcoholic or aqueous alcoholic media, it has been observed that the composition obtained may cause problems of irritation and/or of dehydration of the skin, which results in some discomfort for the user.

The need therefore existed for a cosmetic composition that would transfer only slightly or not at all, or in other words a “non-transferring” composition, which still has good cosmetic properties.

Consequently, there was then proposed, in European Patent Applications EP A 775483 and EP A 793957, a cosmetic composition comprising, in a polymeric system, an aqueous dispersion of particles of film-forming polymer. This system makes it possible to obtain a cohesive film on the substrate on which it is deposited, such film being remarkably resistant to transfer and having good resistance to water.

Nevertheless, it has been observed that, depending on the nature of the polymer employed, removal of the make-up with the usual make-up removers was not very easy, especially using oily make-up removers, because of the good chemical resistance of the polymers to water and/or fatty substances. In addition, in certain cases, the polymer film may be eliminated by means of hydrocarbon solvents, the use of which in cosmetics is not desirable. In other cases, it is necessary to use specific make-up removers, which places constraints upon the user. On the other hand, it has also been observed that the film deposited on the substrate could be slightly sticky, even after drying.

From U.S. Pat. No. 5389363, incorporated herein by reference, there are also known mascara compositions containing polymer gels that can be dispersed in water and silicones. The taught quantity of dispersible polymer is too small to form on the lips or the skin a cohesive film which withstands the movements of the lips and/or of the skin.

### SUMMARY OF THE INVENTION

The present invention provides a cosmetic or dermatological composition which makes it possible to obtain a film of very good stability, which does not transfer and does not stain a substrate with which it may be in contact, and which does not migrate in the course of time, all while having good cosmetic or dermatological properties. In addition, the invention film can be very slightly or not at all sticky, especially on the lips, and furthermore it can be easily removed by means of existing make-up removal products, whether they are aqueous, aqueous alcoholic, or oily.

### EMBODIMENTS OF THE INVENTION

Accordingly, one embodiment of the invention is a cosmetic or dermatological composition suitable for application on the skin and/or the lips, comprising a sufficient quantity of a polymeric system which comprises particles of at least one film-forming polymer in dispersion in a cosmetically or dermatologically acceptable medium and at least one aqueous emulsion of at least one silicone compound.

Another embodiment is a non-transferring make-up or care composition comprising an effective quantity of a polymeric system which comprises particles of at least one film- forming polymer in dispersion in a cosmetically or dermatologically acceptable medium and at least one aqueous emulsion of at least one silicone compound.

Another embodiment is the use of the combination of a polymeric system in effective quantity which comprises particles of at least one film-forming polymer in dispersion in a cosmetically acceptable medium and an aqueous emulsion of at least one silicone compound, in a cosmetic composition suitable for application on the skin and/or the lips, in order to obtain thereon a cohesive film which has very good stability and/or does not transfer and/or does not migrate and/or does not stain and/or does not stick and/or is glossy.

Another embodiment is the use of the combination of a polymeric system in effective quantity which comprises particles of at least one film-forming polymer in dispersion in a cosmetically acceptable medium, and an aqueous emulsion of at least one silicone compound, in a cosmetic composition suitable for application on the skin and/or the lips, to decrease transfer and/or migration of the said composition.

Another embodiment is the use of the combination of a polymeric system in effective quantity which comprises particles of at least one film-forming polymer in dispersion in a dermatologically acceptable medium, and an aqueous emulsion of at least one silicone compound, for the fabrication of a composition intended to treat the skin and/or the lips therapeutically and to form thereon a cohesive film which follows the movements thereof.

These and other embodiments will become clear upon review of the detailed description of the invention, provided below.

### DESCRIPTION OF THE INVENTION

The composition according to the invention makes it possible to obtain a homogeneous cohesive film which has a light texture and remains comfortable to wear throughout the day. The film is soft, supple, elastic and flexible on the skin; it follows the movements of the substrate on which it is deposited without cracking and/or peeling. In particular, it adheres perfectly to the lips of the face.

In addition, the film obtained can be highly glossy or more or less matte, depending on the nature of the constituents of the composition, thus permitting a broader range of glossy or matte make-up products as desired, compared with the prior art.

Another advantage achieved by the present invention is that it is now possible to obtain a non-transferring film which is very gentle and very smooth, non-sticky, without “grease” effect, very resistant to water and very comfortable, in particular for formulating lipsticks, color bases, eyeliners or eye shadows.

The composition according to the invention is readily applicable and spreads easily, especially on the lips of the face. In particular, the composition according to the invention has a particularly interesting application in the field of care and/or make-up of the human skin and/or lips. Thus the composition according to the invention has a preferred application in the field of make-up products for the lips of the face, especially as lipstick. It also has another advantageous application in the field of eye liners, color bases, rouges or eye shadows. It also has the advantage that it can be removed with classical make-up removers, in contrast to prior art compositions.

The composition according to the invention comprises a polymeric system which comprises at least one film-forming polymer in dispersed form, in other words in the form of a dispersion of particles, especially in a cosmetically or dermatologically acceptable medium. Preferably the film-forming polymer has the form of an aqueous dispersion of particles of the polymer.

The aqueous dispersion comprising one or more film-forming polymers can be prepared by the person skilled in the art on the basis of his general knowledge, for example by emulsion polymerization or by making a dispersion of the previously formed polymer.

Film-forming polymers that can be used in the composition according to the present invention include synthetic polymers of the polycondensate or radical type, polymers of natural origin, and mixtures thereof.

Among the polycondensates, examples include the anionic, cationic, nonionic or amphoteric polyurethanes, the polyurethane-acrylates, the polyurethane-polvinlpyrrolidones, the polyester-polyurethanes, the polyether-polyurethanes, the polyureas, the polyurea/polyurethanes and mixtures thereof.

The polyurethane can be, for example, an aliphatic, cycloaliphatic or aromatic polyurethane, polyurea/urethane or polyurea copolymer, containing alone or in a mixture. At least one sequence of straight-chain or branched aliphatic and/or cycloaliphatic and/or aromatic polyester origin, and/or at least one sequence of aliphatic and/or cycloaliphatic and/or aromatic polyether origin, and/or at least one substituted or unsubstituted, branched or unbranched silicone sequence, such as polydimethylsiloxane or polymethylphenylsiloxane, and/or at least one sequence containing fluoro groups, is preferred.

The polyurethanes such as defined in the invention can also be obtained from branched or unbranched polyesters, or from alkyds containing mobile hydrogens, which are modified by reaction with a diisocyanate and a bifunctional organic compound (of dihydro, diamino or hydroxyamino type, for example), additionally containing either a carboxylic acid or carboxylate group or a sulfonic acid or sulfonate group, or even a neutralizable tertiary amine group or a quaternary ammonium group.

Also useful are the polyesters, the polyester amides, the fatty-chain polyesters, the polyamides and the epoxy ester resins.

The polyesters can be obtained in known manner by polycondensation of aliphatic or aromatic diacids with aliphatic or aromatic diols or polyols. As aliphatic diacids there can be used succinic acid, glutaric acid, adipic acid, pimelic acid, suberic acid or sebacic acid. As aromatic diacids there can be used terephthalic or isophthalic acid or even a derivative such as phthalic anhydride. As aliphatic diols there can be used ethylene glycol, propylene glycol, diethylene glycol, neopentyl glycol, cyclohexanedimethanol, and 4,4′-(1-methylopropylidene) bisphenol. As polyols there can be used glycerol, pentaerythritol, sorbitol, and trimethylolpropane.

The polyester amides can be obtained in a way analogous to that for the polyesters by polycondensation of diacids with diamines or amino alcohols. As diamine there can be used ethylenediamine, hexamethylenediamine, meta-phenylenediamine or para-phenylenediamine. As amino alcohol there can be used monoethanolamine.

Examples of the monomer carrying the anionic group that can be used during polycondensation include dimethylolpropionic acid, trimellitic acid or a derivative such as trimellitic anhydride, the sodium salt of 3-sulfopentanediol acid, and the sodium salt of 5 -sulfo-1,3-benzenedicarboxylic acid.

The fatty-chain polyesters can be obtained by the use of fatty-chain diols during polycondensation.

The epoxy ester resins can be obtained by polycondensation of fatty acids with a condensate at the α,ω-diepoxy ends.

The radical polymers can be obtained especially from acrylic and/or vinyl copolymers. Preferably there are used anionic radical polymers. Examples of the monomer carrying the anionic group that can be used during radical polymerization include acrylic acid, methacrylic acid, crotonic acid, maleic anhydride, and 2-acrylamido-2-methylpropanesulfonic acid.

The acrylic polymers can result from the copolymerization of monomers chosen from among the esters and/or amides of acrylic acid or methacrylic acid. As example of monomers of ester type there can be cited methyl methacrylate, ethyl methacrylate, butyl methacrylate, isobutyl methacrylate, 2-ethylhexyl methacrylate, and lauryl methacrylate. As example of monomers of amide type there can be cited N-t-butylacrylamide and N-t-octylacrylamide.

Preferably there are used acrylic polymers obtained by copolymerization of monomers of ethylene-type unsaturation containing hydrophilic groups, preferably of nonionic nature, such as hydroxyethyl acetate, 2-hydroxypropyl acrylate, hydroxyethyl methacrylate, 2-hydroxypropyl methacrylate.

The vinyl polymers can result from homopolymerization or copolymerization of monomers chosen from among the vinyl esters, styrene or butadiene. As examples of vinyl esters there can be cited vinyl acetate, vinyl neodecanoate, vinyl pivalate, vinyl benzoate and vinyl t-butylbenzoate.

There can also be used acrylate/silicone copolymers or even nitrocellulose/acrylate copolymers.

The polymers of natural origin, possibly modified, can be chosen from among shellac resin, sandarac gum, the dammars, the elemis, the copals, the cellulose derivatives and mixtures thereof.

There can also be cited the polymers resulting from the radical polymerization of one or more radical monomers in the interior and/or partly at the surface of preexisting particles of at least one polymer chosen from among the group comprising the polyurethanes, the polyureas, the polyesters, the polyester amides and/or the alkyds. These polymers are generally referred to as hybrid polymers.

The composition according to the invention can also contain water-soluble polymers such as protein derivatives of animal or vegetable origin and more particularly hydrolyzed keratins and sulfonic keratins, polyvinylpyrrolidone, vinyl copolymers such as the copolymer of methyl vinyl ether and maleic anhydride or the copolymer of vinyl acetate and crotonic acid, glycoaminoglycans, hyaluronic acid and its derivatives, acrylate polymers, polysaccharides, cellulose polymers and derivatives thereof. These polymers will in particular be used if there is desired more or less sensitive elimination of the film with water.

In order to improve the film-forming character of a polymer, for example by lowering its glass transition temperature, it is possible to add to the polymer system a blending agent, which can be chosen from among the known blending agents.

When an aqueous dispersion of polymer particles is employed, the content of dry material of the aqueous dispersion can be on the order of 5 to 60%, and preferably 30 to 50% by weight; based on total weight dispersion.

The size of the polymer particles in aqueous dispersion can be between 10 and 500 nm, and is preferably between 20 and 150 nm, thus making it possible to obtain a film having remarkable gloss.

The composition can comprise from 2 to 60%, better 5 to 60% and preferably 2 to 30% by weight of dry material of film-forming polymers based on total weight. More generally, the total quantity of polymer is preferably present in a quantity sufficient to form on the skin and/or on the lips a cohesive film, capable of following the movements of the skin and/or of the lips without peeling or cracking. It is within the skill of the ordinary artisan to determine the amount of film forming polymer, etc. to obtain the results stated herein regarding film formation, gloss, transferability, etc.

The composition can also comprise at least one hydrophilic or hydrophobic plasticizer, chosen for its compatibility with the polymer or polymers, and in quantity such that it does not impair the sensitivity of the film to water. The said plasticizer can be chosen from among all the compounds known to the person skilled in the art as being capable of performing the desired function. This agent can be water-soluble or insoluble in water, and if necessary may have the form of an aqueous dispersion.

In particular, there can be cited, alone or as mixtures, the usual plasticizers such as:

the glycols and their derivatives such as diethylene glycol ethyl ether, diethylene glycol methyl ether, diethylene glycol butyl ether or even diethylene glycol hexyl ether, ethylene glycol ethyl ether, ethylene glycol butyl ether, and ethylene glycol hexyl ether,

the glycerol esters,

the derivatives of propylene glycol and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether, propylene glycol butyl ether,

esters of acids, especially carboxylic, such as citrates, phthalates, adipates,

carbonates, tartrates, phosphates, sebacates,

oxyethylene derivatives such as the ethoxylated oils, especially the vegetable oils such as castor oil; the silicone oils.

The quantity of plasticizer can be chosen by the person skilled in the art on the basis of his general knowledge, so as to obtain a film having the desired mechanical properties, while retaining cosmetically acceptable properties in the composition.

According to the invention, the term “polymeric system” will be used to mean the mixture comprising the film-forming polymer or polymers and any water-soluble polymers, blending agents and plasticizers, etc., that may be used; this polymeric system must be adapted to form a supple, flexible, cohesive film on the substrate on which it is deposited, in order to follow the movements of the substrate (lips or skin) on which it is deposited.

Preferably the said film will satisfy at least one of the following physicochemical conditions under the measurement conditions defined before the examples:

a Young's modulus of lower than about 200 MPa, preferably lower than about 100 MPa, and preferentially lower than 80 MPa, and/or

an elongation greater than about 200%, and preferentially greater than 300%, and/or

a hardness lower than 110, preferably lower than 70, more preferentially lower than

The composition according to the invention also comprises an aqueous emulsion of at least one silicone compound.

The aqueous emulsion is an emulsion of the oil-in-water type in which water is the continuous phase and silicone is the dispersed phase. This emulsion can be stabilized by an emulsifying system of the usual type.

The emulsion, in which the size of the silicone droplets can be smaller than 1 μm, can also have the form of a transparent microemulsion having a droplet size on the order of 10 to 80 nm. The silicone microemulsions are stable emulsions of colloidal particles.

The silicone compounds present in the dispersed phase of the emulsion are preferably polyorganosiloxanes, which can have the form of oils, gums, resins or waxes.

The silicone compounds can generally be readily emulsified in an aqueous medium.

The silicone gums, waxes and resins can be added to the composition in the form of an emulsion in water, or in a mixture with liquid silicones in which they are solubilized. In general, the silicone compounds are preferably polymers containing repeating units of formula: Rₙ,SiO_{(4-n)/2}

The substituents R present in these repeating units are organic groups, which can be identical or different. In addition, one and the same compound can contain different repeating units.

The repeating units corresponding to n=2 generally endow the compound with a straight-chain or cyclic structure in which the chain comprises siloxane bonds. In the case of a straight-chain polymer, units corresponding to n=3 comprise the terminal groups.

In addition, the polyorganosiloxanes can contain cross-linking units interposed between the repeating units. These cross-linking units correspond to the formula hereinabove with n=1 or n=0.

In the repeating units (n=2) and the cross-linking units corresponding to n=1, the groups R can represent especially alkyl, cycloalkyl or aryl groups and can also contain functional groups such as ethers, amines, carboxyls, hydroxyls, thiols, esters, sulfonates, and sulfates.

The alkyl groups have, for example, 1 to 20 carbon atoms; the cycloalkyl groups have, for example, 5 or 6 members; the aryl groups are especially phenyl groups.

In the case of terminal groups corresponding to n=3, one of the groups R attached to the terminal silicon can in addition represent another group, such as an OH group.

Among the silicone compounds suitable for use in the present invention there can be cited the non-volatile silicones chosen in particular from among the polyorganosiloxanes and especially the polyalkylsiloxanes, the polyarylsiloxanes, the polyalkylarylsiloxanes, the polyether siloxane copolymers, which may or may not be organomodified, the silicone gums and resins, the polysiloxanes modified by organofunctional groups as well as mixtures thereof.

The silicone compounds are chosen more particularly from among the polyalkylsiloxanes, among which there can be cited mainly the straight-chain polydimethylsiloxanes with trimethylsilyl terminal groups having a viscosity of 5.10⁻⁶ to 2.5 m²/s at 25° C. preferably of 1.10⁻⁵ to 1 m²/s at 25° C. and especially the following commercial products:

the series 47 and 70047 SILBIONE oils sold by RHONE POULENC, such as oil 47V500000.

the series 200 oils of DOW CORNING,

the VISCASIL oils of GENERAL ELECTRIC,

certain SF series oils (SF96, SF 18) of GENERAL ELECTRIC.

There can also be cited the straight-chain polydimethylsiloxanes with dimethylsilanol terminal groups, such as the 48 series oils of RHONE POULENC.

There can also be cited the products sold under the names ABIL WAX 9800 and 9801 of the GOLDSCHMIDT Co., which are C₁-C₂₀ polyalkylsiloxanes.

Among the polyalkylarylsiloxanes there can be cited the straight-chain or branched polydimethylmethylphenylsiloxanes and the polydimethyldiphenylsiloxanes having a viscosity of 1.10⁻⁵ to 5.10⁻²m²/s at 25° C. and especially the following commercial products:

the series 70641 SILBIONE oils of RHONE POULENC,

the series RHODORSIL 70633 and 763 oils of RHONE POULENC,

DC 556 Fluid oil of DOW CORNING,

the series PK silicones of BAYER, such as the product PK20,

the series PN and PH silicones of BAYER, such as the products PN1000 and PH1000,

certain oils of the SF series of GENERAL ELECTRIC, such as SF1023, SF 1154, SF1250 and SF1265.

The silicone gums that can be used in accordance with the present invention are especially polydiorganosiloxanes having high molecular weights, preferably of between 200,000 and 1,000,000. They can be used alone or in mixtures in a solvent, which can be chosen from among the volatile silicones, the polydimethylsiloxanes (PDMS), the polyphenylmethylsiloxanes (PPMS), the isoparaffins, methylene chloride, pentane, dodecane, tridecane, tetradecane or mixtures thereof.

There can be cited more particularly the following products: the polydimethylsiloxane/methyvinylsiloxane, polydimethylsiloxane/diphenylsiloxane, polydimethylsiloxane/phenylmethylsiloxane, polydimethylsiloxane/diphenylsiloxane/ methyvinylsiloxane gums.

In particular, there can be employed mixtures such as:

mixtures formed from a polydimethylsiloxane hydroxylated at the chain end (dimethiconol according to the CTFA) and from a cyclic polydimethylsiloxane (cyclomethicone according to the CTFA) such as the product Q2-1401 sold by the DOW CORNING Co.,

mixtures formed from a polydimethylsiloxane gum with a cyclic silicone oil such as the product SF1214 of GENERAL ELECTRIC (which is a mixture of dimethicone gum having a molecular weight of 500,000 solubilized in decamethylcyclopenta-siloxane),

mixtures of two PDMS of different viscosities, especially of a PDMS gum and a PDMS oil, such as the product SF1236 of GENERAL ELECTRIC Co. (which is a mixture of 15% of dimethicone gum having a molecular weight of 500,000 and viscosity of 20 m²/s, and of 85% of SF96 oil having a viscosity of 5.10⁻⁶ m²/s).

The organopolysiloxane resins that can be used according to the invention are especially cross-linked siloxane systems containing R₂SiO, RSiO_{3/2}, and SiO₂ units. Among these compounds, the more particularly preferred products are those in which R designates a C₁−C₈ alkyl or phenyl.

Among these resins there can be cited the product sold under the name DOW CORNING 593 or those sold under the names Silicone Fluid SS4230 and SS4267 by the GENERAL ELECTRIC Co., which are of the dimethyl/trimethylsiloxane type.

The organomodified silicones are silicone compounds such as defined hereinabove, additionally containing in their structure one or more organic functional groups fixed directly to the siloxane chain or fixed through a hydrocarbon radical.

There can be cited, for example, the silicones containing:

polyethyleneoxy and/or polypropyleneoxy groups, possibly containing alkyl groups such as:

dimethicone copolyols, and especially that sold by the DOW CORNING Co. under the name DC 1248,

alkylmethicone copolyols, and especially C₁₂ alkylmethicone copolyol sold by the DOW CORNING Co. under the name Q2-5200,

SILWET L 722, L 7500, L 77, and L 711 oils of the UNION CARBIDE Co.,

amine groups, possibly substituted, such as the products sold under the names GP4 Silicone Fluid and GP7100 by the GENESEE Co. or the products sold under the names Q2-8220 and DC929 by the DOW CORNING Co. The substituted amine groups are in particular C₁-C₄ aminoalkyl groups,

thiol groups such as in GP 72 A and GP 71 of GENESEE,

carboxylate groups such as in the products described in European Patent EP 186507 of CHISSO CORPORATION,

alkoxylated groups, such as the products sold under the name Silicone polymer F-755 by SWS SILICONES, or ABIL WAX 2428, 2434 and 2440 by the GOLDSCHMIDT Co.

hydroxyl groups, such as the polyorganosiloxanes with hydroxyalkyl function, described in particular in French Patent Application 85FR-16334,

acyloxyalkyl groups, such as the polyorganosiloxanes described in French Patent Application 88FR-17433. These compounds can be prepared by esterification of polyorganosiloxanes with hydroxyalkyl function,

anionic groups of carboxylic type, such as the alkylcarboxylic groups; 2-hydroxyalkylsulfonate or 2-hydroxyalkylthiosulfate,

fluoro groups.

Among the silicone compounds suitable for use in the present invention there can also be cited the volatile (at room temperature and atmospheric pressure) silicones, which generally have a boiling point located between 60 and 260° C. and a viscosity of <8 cts and, in particular:

cyclic silicones containing 3 to 7 silicon atoms and preferably 4 to 6 silicon atoms, such as octamethylcyclotetrasiloxane, decamethylcyclopentasiloxane, dodecamethylcyclohexasiloxane and mixtures thereof.

There can also be cited the cyclocopolymers such as dimethylsiloxane-methylalkylsiloxane and especially the volatile silicone FZ 3109 sold by UNION CARBIDE. There can also be cited the mixtures of cyclic silicones with compounds derived from silicon, such as the mixture of octamethylcyclotetrasiloxane and tetratrimethyl-silylpentaerythritol (50/50) and the mixture of octamethylcyclotetrasiloxane and 1,1′-oxy(2,2,2′,2′,3,3′-) hexatrimethylsilyloxy)bisneopentane,

straight-chain volatile silicones containing 2 to 9 carbon atoms and having a viscosity of lower than or equal to 5.10⁻⁶ m²/s at 25° C., such as hexamethyl-disiloxane or decamethyltetrasiloxane. Silicones in this class are also described in the article published in Cosmetic and Toiletries, Vol. 91, Jan. 76, pp. 27-32 entitled “Volatile silicone fluids for cosmetics”.

The person skilled in the art will be able easily to prepare the aqueous emulsion of silicone compounds on the basis of his general knowledge.

Among the commercially available aqueous silicone emulsions there can be cited:

polydimethylsiloxane emulsions (SM2162 of General Electric),

stearyldimethicone emulsions (SLM23032 of Wacker),

amodimethicone microemulsions (Microemulsion 71827 of Rh6ne-Poulenc),

cationic microemulsions of polydimethylsiloxane with aminoethylaminopropyl groups (DC939 of Dow Corning),

polydimethylsiloxane microemulsions (SILTECH MFF 5015-70 of Siltech or DC2- 1281 of Dow Coming).

The silicone compounds can be present in the composition in a proportion of 0.1% to 30% by weight of dry silicone material based on total weight of composition, preferably in a proportion of 5% to 30% by weight.

Furthermore, in a preferred embodiment of the invention, the composition can comprise an aqueous dispersion of wax, preferably an aqueous microdispersion of wax. The waxes can also be incorporated in the form of particles dispersed in water. By wax microdispersion there is understood an aqueous dispersion of wax particles in which the size of the said wax particles is smaller than or equal to approximately 1 micron.

The wax microdispersions are stable dispersions of colloidal particles of wax, and are described in particular in “Microemulsions Theory and Practice”, L.M. Prince, Ed., Academic Press (1977), pages 21-32; incorporated herein by reference.

In particular, these wax microdispersions can be obtained by melting the wax in the presence of a surfactant, and possible a portion of the water, followed by progressive addition of hot water with agitation. There is observed the intermediate formation of an emulsion of the water-in-oil type, followed by phase inversion, ultimately yielding an emulsion of the oil-in-water type. On cooling, there is obtained a stable microdispersion of solid colloidal particles of wax.

The particles of the wax microdispersion preferably have mean dimensions smaller than 1 μm. preferably smaller than 0.5 μm. These particles are comprised substantially of a wax or a mixture of waxes. Nevertheless, they can comprise a minority proportion of oily and/or pasty fatty additives, a surfactant and/or a usual fat-soluble additive/active agent.

The waxes suitable for use in the compositions according to the invention are chosen from among the waxes of animal, vegetable, mineral or synthetic origin and mixtures thereof which are solid and rigid at room temperature. Preferably the waxes used in the composition can have a melting point higher than about 45° C. and in particular higher than 55 °, and/or a needle penetration index at 25° C. of preferably between 3 and 40, measured according to American Standard ASTM D 5 or according to French Standard NFT 004.

There can be cited in particular beeswax, wool wax, Chinese insect wax, rice wax, carnauba wax, candellila wax, ouricury wax, esparto wax, cerin, sugarcane wax, Japan wax, sumac wax, montan wax, microcrystalline waxes, paraffin waxes, ozokerite, polyethylene waxes, waxes obtained by Fischer-Tropsch synthesis and the waxy copolymers as well as esters thereof.

There can also be cited the waxes obtained by catalytic hydrogenation of animal or vegetable oils having C8-C32 straight-chain or branched fatty chains. Among those, there can be cited in particular hydrogenated jojoba oil, hydrogenated sunflower oil, hydrogenated castor oil, hydrogenated copra oil and hydrogenated lanolin oil.

There can also be cited the silicone waxes.

It is also possible to use commercial mixtures of self-emulsifying waxes containing a wax and surfactants. There can be used, for example, the wax sold under the name “Cire Auto Lustrante OFR” by Tiscco, which contains carnauba and paraffin waxes in combination with nonionic surfactants, or the self-emulsifying wax sold under the name “Cerax A.O. 28/B” by La Ceresine, which contains esparto wax in combination with a nonionic surfactant. These commercial mixtures make it possible to prepare microdispersions of waxes by simple addition of water.

There can also be cited the “Aquacer” products of Byk Cera, and in particular: the 25 mixture of synthetic and natural waxes with anionic emulsifier (Aquacer 520), the polyethylene wax with nonionic emulsifier (Aquacer 514 or 513), the polymeric wax with anionic emulsifier (Aquacer 511). There can also be cited the mixture of polyethylene waxes and paraffin with nonionic emulsifier: “Jonwax 120” of Johnson Polymer.

Preferably at least one of the waxes is fluorinated.

By fluorinated wax there is understood a wax whose chemical structure comprises at least one fluoro or perfluoro group, or a mixture of waxes, at least one of which comprises a fluoro or perfluoro group.

There can be cited the micronized mixtures of polyethylene wax and PTFE (polytetrafluoroethylene) wax.

Among the microdispersions of commercial fluorinated waxes that can be used according to the invention there can be cited “Microdispersion 411” of MicroPowders, which is a mixture of polyethylene, polytetrafluoroethylene and acrylate polymer waxes. As fluorinated wax there can be cited the product “Aquapolyfluo 411” of MicroPowders, which is a mixture of polyethylene wax and polytetrafluoroethylene.

The composition according to the invention can comprise 0 to 50% by weight of dry wax material, more particularly 0.1 to 30% by weight based on total weight of composition.

The aqueous (micro)dispersion preferably comprises between 10 and 70% by weight of dry wax material.

The composition according to the invention can comprise a sufficient quantity of surfactant to make it possible to obtain a wax microdispersion and/or a silicone emulsion, as well as a stable final composition. In particular, it can comprise 0.01 to 30% by weight of usual surfactant, which can be chosen from among the following compounds:

the anionic surfactants, especially salts of fatty acids, which may be unsaturated, having 12 to 18 carbon atoms, for example; alkali metal salts or salts of organic bases with alkylsulfuric and alkylsulfonic acids having 12 to 18 carbon atoms or alkylarylsulfonic acids whose alkyl chain contains 6 to 18 carbon atoms; ether sulfates,

the nonionic surfactants, especially polyalkoxylated and/or polyglycerated surfactants, and in particular fatty acids or fatty acid amides; fatty alcohols or alkylphenols; esters of fatty acids and polyols; alkanediols and alkyl ethers of alkanediols. There can also be cited the alkylcarbamates of triglycerol, the ethoxylated or propoxylated derivatives of lanolin alcohols, fatty acids of lanolin, or mixtures thereof,

cationic surfactants, especially quaternary ammonium derivatives.

The wax or mixture of waxes can be combined with one or more fatty additives (oily and/or pasty). There can be cited especially the vegetable oils such as sunflower oil, jojoba oil; the mineral oils such as paraffin oil; the silicone oils; vaseline, lanolin; the fluorinated oils; the hydrocarbonated oils with perfluoro group; the esters of fatty alcohols.

It is possible to introduce in addition into the silicone and/or waxy phase fat-soluble active ingredients such as UV screens, fat-soluble vitamins, and fat-soluble cosmetic active agents.

The compositions according to the invention can have different forms containing an aqueous phase and in particular the form of oil-in-water or water-in-oil emulsions or the form of aqueous dispersions. In particular, the quantity of water of composition represents from 10 to 97.9%, and better from 30 to 70% of the total weight of the composition.

According to a preferred embodiment, they have the form of oil-in-water emulsions, which can comprise at least one surfactant, especially of anionic or nonionic type, in a proportion of between 2 and 30% by weight relative to the total weight of the composition.

The composition can also comprise a coloring material used in the usual manner in the field of cosmetics and make-up, such as a water-soluble coloring agent and/or a pigment.

The pigments can be present in the composition in a proportion of 0 to 20% by weight of the final composition, and preferably in a proportion of 1 to 5%. They can be white or colored, mineral and/or organic, of usual or nanometer size. Among the mineral pigments and nanopigments there can be cited the oxides of titanium, zirconium or cerium as well as the oxides of zinc, iron or chromium, and Prussian blue. Among the organic pigments, there can be cited carbon black and the lakes of barium, strontium, calcium, and aluminum.

Among the water-soluble colorants there can be cited the usual colorants of the field under consideration, such as the disodium salt of ponceau, the disodium salt of alizarine green, quinoline yellow, the trisodium salt of amaranth, the disodium salt of tartrazine, the monosodium salt of rhodamine, the disodium salt of fuchsine, xanthophyll, and mixtures thereof.

There can also be added to the composition according to the invention any known additive such as thickeners, for example clays, gums, silicas, cellulose derivatives, a synthetic polymer such as an acrylate polymer or addition polymer of the polyurethane type; a natural gum such as xanthan gum; spreading agents; dispersants; preservatives; antifoaming agents; wetting agents; UV screens; perfumes; fillers; cosmetic active agents such as humectants, vitamins and derivatives thereof; biological materials and derivatives thereof; dermatological active agents for the purpose of endowing the composition with therapeutic care and/or treatment properties for topical application.

The person skilled in the art is capable of choosing such possible additive or additives and/or the quantity thereof so that the advantageous properties of the composition according to the invention are not altered substantially or at all by the envisioned addition.

This composition must of course be capable of being deposited on a substrate such as the skin and/or the lips.

The composition according to the invention can have the form of a fluid, gel, semisolid, supple paste or even solid such as a stick or wand. In the case of a non-solid formula, the composition according to the invention can have a viscosity ranging from 0.05 Pa.s to 40 Pa.s (50 cPs to 40000 cPs), and especially from 0.05 Pa.s to 10 Pa.s measured at 25° C. using a portable Brookfield 4 LVT instrument.

In particular, it can be applied as a make-up product or in the preparation of such a product, especially as lipstick, color base, rouge or eye shadow, or even eye liner. There can also be envisioned an application in the field of care compositions, sun-protection compositions or self-acting tanning compositions, to be applied on the skin and/or the lips.

Removal of the make-up compositions according to the invention is achieved easily with conventional make-up removers and/or make-up removal oils. It can also be achieved by means of warm soapy water or standard two-phase (oil/water) make-up removers.

The invention will be illustrated in more detail in the following examples.

A/ Measurement Of Elongation

The elongation of the film obtained is measured in accordance with the ASTM Standards, Volume 06.01, D 2370-92: “Standard Test Method for Tensile Properties of Organic Coatings”.

B/ Measurement Of Hardness

The hardness of the film is measured in accordance with ASTM D 43-66 or with French Standard NF-T 30-016 (October 1981), using a Persoz pendulum.

The film deposited on the substrate must have a thickness of about 300 microns before drying. After drying for 24 hours at 30° C. under a relative humidity of 50%, there is obtained a film having a thickness of about 100 microns; its hardness is then measured at 30° C. and 50% relative humidity.

C/ Measurement Of Young's Modulus (or modulus of elasticit)

Young's modulus (modulus of elasticity) is measured in accordance with the ASTM 10 Standards, Volume 06.01, D 2370-92: “Standard Test Method for Tensile Properties of Organic Coatings”.

The film deposited on the substrate must have a thickness of about 300 microns before drying. After drying for 7 days at 21° C. under a relative humidity of 50%, there is obtained a film having a thickness of about 100 microns.

The measured samples have a width of 5 mm and a thickness of 100 microns. The distance between the jaws is 25 mm. The rate of elongation is 1000 mm per minute.

### Example 1

There were prepared aqueous compositions comprising:

4.8% of pigment paste comprising 10 to 16% by weight of glycerine, so as to obtain 2% by weight of pigments in the composition, and

the following compounds:

| | |
|---|---|
| composition A | aqueous emulsion of PDMS (47.5 wt %) |
| composition B | aqueous dispersion of acrylate polymer (42.7 wt %) |
| composition C | aqueous emulsion of PDMS (20.2 wt %) + aqueous |
| | dispersion of acrylate polymer (18.2 wt %) according |
| | to the invention. |

The compositions were applied on the lips of 10 persons, who made a sensory evaluation according to the following criteria: gloss, transfer, stickiness, removal with cold water, removal with warm soapy water (1% of teepol in water) and removal with oil.

The following results were obtained:

| | Gloss | Transfer | Stickiness |
|---|---|---|---|
| Composition A | +++ | ++ | ++ |
| Composition B | +++ | −− | −− |
| Composition C | +++ | −− | + |
| | Removal with | Removal with | Removal with |
| | cold water | warm soapy water | oil |
| Composition A | +++ | +++ | +++ |
| Composition B | −− | −− | +/− |
| Composition C | −− | ++ | +++ |

It was therefore observed that composition C according to the invention can be removed easily using a conventional oily make-up remover or warm soapy water.

In addition, it was observed that composition C according to the invention did not transfer at all and was only slightly sticky when applied to the lips, and was also glossy.

### Example 2

There was prepared a composition comprising the following constituents:

| | |
|---|---|
| aqueous dispersion of acrylate polymer | 13 wt % |
| (45% dry material) | |
| aqueous silicone emulsion (50% dry | 14.5 wt % |
| material) | |
| aqueous dispersion of polymeric wax | 7 wt % |
| (25% dry material) | |
| pigment | 2% |
| glycerine | 1.25% |
| gelling agent | 0.9% |
| water | made up to 100% |

The wax was first dispersed in water, and then the other compounds were added. There was obtained a fluid lipstick composition which was applied easily on the lips, forming a glossy film, which was only slightly sticky, did not transfer and was resistant to water.

It was removed easily using a conventional make-up remover oil.

### Example 3

There was prepared a composition comprising the following constituents:

| | |
|---|---|
| aqueous dispersion of acrylate polymer | 18 wt % |
| (45% dry material) | |
| aqueous silicone emulsion (50% dry | 20.5 wt % |
| material) | |
| pigment | 2% |
| glycerine | 1.25% |
| gelling agent | 1.2% |
| water | made up to 100% |

There was obtained a fluid lipstick composition which was applied easily on the lips, forming a glossy film, which did not transfer and was removed easily using conventional make-up remover oils.

French patent application No.97 15414 filed Dec. 5, 1997, is incorporated herein by reference.

Obviously, numerous modifications and variations of the present invention are possible in light of the above teachings. It is therefore to be understood that within the scope of the appended claims, the invention may be practiced otherwise than as specifically described herein.

1. A cosmetic or dermatological composition suitable for application on the skin and/or the lips, comprising:
a) a polymeric system comprising particles of at least one film-forming polymer dispersed in a cosmetically or dermatologically acceptable medium; and
b) an aqueous emulsion of at least one silicone compound.

2. The composition according to claim 1, wherein the film-forming polymer is present in the composition in the form of an aqueous dispersion of particles.

3. The composition according to claim 2, wherein the particles have a size of from 10 to 500 nm.

4. The composition according to claim 1, wherein the film-forming polymer is selected from the group consisting of synthetic polycondensate or radical polymers, polymers of natural origin, and mixtures thereof.

5. The composition according to claim 1, wherein the film-forming polymer is selected from the group consisting of anionic, cationic, nonionic or amphoteric polyurethanes; polyurethane-acrylates; polyurethane-polyvinylpyrrolidones; polyester-polyurethianes; polyether-polyurethanes; polyureas; polyurea/polyurethanes; polyesters; polyester amides; fatty-chain polyesters; polyamides; epoxy ester resins; acrylic and/or vinyl polymers or copolymers; acrylate/silicone copolymers; nitrocellulose/acrylate copolymers; shellac resin, sandarac gum, dammars, elemis, copals, cellulose derivatives; hybrid polymers; and mixtures thereof.

6. The composition according to claim 1, formulated such that it forms on the skin and/or the lips a film which does not transfer and/or is resistant to water and/or is glossy and/or does not stain and/or does not migrate and/or is stabile.

7. The composition according to claim 1, in which the polymeric system is capable of forming a film on the skin and/or lips which satisfies at least one of the following conditions:
1) a Young's modulus of lower than 200 MPa,
2) an elongation greater than 200%,
3) a hardness lower than 110.

8. The composition according to claim 1, in which a quantity of dry materials is from 2 to 60% by weight relative to the total weight of the composition.

9. The composition according to claim 1, in which the aqueous emulsion of at least one silicone compound has a droplet size smaller than 1 μm.

10. The composition according to claim 1, in which the at least one silicone compound is selected from the group consisting of polyorganosiloxanes, polyarylsiloxanes, polyalkylarylsiloxanes, polyether siloxane copolymers which may or may not be organomodified, silicone gums and resins, polysiloxanes modified by organofunctional groups, volatile silicones, and mixtures thereof.

11. The composition according to claim 1, in which the at least one silicone compound is selected from the group consisting of the straight-chain polydimethylsiloxanes with trimethylsilyl terminal groups; straight-chain polydimethylsiloxanes with dimethylsilanol terminal groups; polydimethylmethylphenylsiloxanes; and polydimethyldiphenylsiloxanes.

12. The composition according to claim 1, in which the silicone compound of the aqueous emulsion is present in the composition in an amount of 0.1% to 30% by weight of total dry silicone material.

13. The composition according to claim 1, further comprising an aqueous dispersion of wax.

14. The composition according to claim 13, in which the wax is selected from the group consisting of beeswax, wool wax, Chinese insect wax, rice wax, carnauba wax, candellila wax, ouricury wax, esparto wax, cerin, sugarcane wax, Japan wax, sumac wax, montan wax, microcrystalline waxes, paraffin waxes, ozokerite, polyethylene waxes, waxes obtained by Fischer-Tropsch synthesis and the waxy copolymers as well as esters thereof, waxes obtained by catalytic hydrogenation of animal or vegetable oils having C8-C32 straight-chain or branched fatty chains, silicone waxes, and fluorinated waxes.

15. The composition according to claim 1, having the form of a lipstick, color base, rouge, eye shadow, eye liner, a skin or lip care composition, or a sun-protection or self-acting tanning composition.

16. A composition comprising a polymeric system which comprises particles of at least one film-forming polymer dispersed in a cosmetically or dermatologically acceptable medium, and at least one aqueous emulsion of at least one silicone compound.

17. A method of caring for or making up the lips and/or the skin, comprising applying to the lips and/or the skin the composition of claim 1.

18. A method for preparing a composition, comprising mixing together particles of at least one film-forming polymer and an aqueous emulsion of at least one silicone compound.
